SEQUENCE LISTING (4 sequences)

SEQ ID NO: 1 - DNA, 729 bp, Homo sapiens
atgtctggggatgcgaccaggaccctggggaaaggaagccagcccccagggccagtcccggaggggctgatccgcatctacagcatgaggttctgcccctattctcacaggacccgcctcgtcctcaaggccaaagacatcagacatgaagtggtcaacattaacctgagaaacaagcctgaatggtactatacaaagcacccttttggccacattcctgtcctggagaccagccaatgtcaactgatctatgaatctgttattgcttgtgagtacctggatgatgcttatccaggaaggaagctgtttccatatgacccttatgaacgagctcgccaaaagatgttattggagctattttgtaaggtcccacatttgaccaaggagtgcctggtagcgttgagatgtgggagagaatgcactaatctgaaggcagccctgcgtcaggaattcagcaacctggaagagattcttgagtatcagaacaccaccttctttggtggaacctgtatatccatgattgattacctcctctggccctggtttgagcggctggatgtgtatgggatactggactgtgtgagccacacgccagccctgcggctctggatatcagccatgaagtgggaccccacagtctgtgctcttctcatggataagagcattttccagggcttcttgaatctctattttcagaacaaccctaatgcctttgactttgggctgtaa

SEQ ID NO: 2 - protein, 242 aa, Homo sapiens
MSGDATRTLGKGSQPPGPVPEGLIRIYSMRFCPYSHRTRLVLKAKDIRHEVVNINLRNKPEWYYTKHPFGHIPVLETSQCQLIYESVIACEYLDDAYPGRKLFPYDPYERARQKMLLELFCKVPHLTKECLVALRCGRECTNLKAALRQEFSNLEEILEYQNTTFFGGTCISMIDYLLWPWFERLDVYGILDCVSHTPALRLWISAMKWDPTVCALLMDKSIFQGFLNLYFQNNPNAFDFGL

SEQ ID NO: 3 - DNA, 747 bp, Murine
atgtccggagatttgtcaagatgcttgggaaaaggaagctgtcccccagggccggtccctgagggtgtgatccgaatctacagcatgaggttctgcccctactcgcacagggcacgcctggttctcaaggccaaaggcatcaggcatgaagtgatcaatattaacctgaaaagcaagcctgactggtactatacaaagcatccttttggccaaattcctgtcttggagaacagccagtgtcagctggtctatgaatctgtcattgcttgtgagtacctggatgacgtctacccgggaagaaagctgtttccgtatgacccgtatgaacgagctcgccagaagatgttattggagctattctgtaaggtcccgcctttaagcaaggaatgtctgatagcgctgagatgcggaagagactgtacggatctgaaggtcgccctgcgtcaggagttgtgcaacatggaagagattcttgaatatcagaacactaccttcttcggcggagactgtatatccatgattgattacctcgtctggccctggtttgagcgcctggacgtatatggactggctgactgcgtgaatcacaccccgatgctgcggctctggatagcctccatgaagcaggaccctgcagtgtgtgctctgcacactgataagagcgtcttcctgggcttcttgaatctctatttccaaaacaacccttgtgcctttgattttgggctgtgtaacccaatcatacgataa

SEQ ID NO: 4 - protein, 248 aa, Murine
MSGDLSRCLGKGSCPPGPVPEGVIRIYSMRFCPYSHRARLVLKAKGIRHEVININLKSKPDWYYTKHPFGQIPVLENSQCQLVYESVIACEYLDDVYPGRKLFPYDPYERARQKMLLELFCKVPPLSKECLIALRCGRDCTDLKVALRQELCNMEEILEYQNTTFFGGDCISMIDYLVWPWFERLDVYGLADCVNHTPMLRLWIASMKQDPAVCALHTDKSVFLGFLNLYFQNNPCAFDFGLCNPIIR

(19) United States
(12) Patent Application Publication
(10) Pub. No.: US 2005/0064519 A1
(43) Pub. Date: Mar. 24, 2005

(54) Methods of using GST-Omega-2

(75) Inventors: Christopher A. Gabel, Ledyard, CT (US); Kieran F. Geoghegan, Mystic, CT (US); Jose R. Perez, Salem, CT (US)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 10/818,204

(22) Filed: Apr. 5, 2004

(30) Foreign Application Priority Data
Sep. 22, 2003 (WO) PCT/IB03/04384

(51) Int. Cl. G01N 33/53; G01N 33/567

(52) U.S. Cl. 435/7.2

(57) ABSTRACT

The present invention relates to GST-Omega-2 and methods of using this diarylsulfonylurea binding protein (DBP) as a therapeutic target for the identification of agents that suppress the release of inflammatory mediators such as interleukin IL-1 and IL-18.

This application claims the benefit of U.S. Provisional Application No. 60/416,044, filed on Oct. 3, 2002 and PCT International Application No. PCT/IB 03/04384, filed on Sep. 22, 2003 and incorporated herein by reference in its entirety.

### REFERENCE TO RELATED APPLICATION

This application is related to application Ser. No. 09/387,372, filed Aug. 31, 1999, now published as U.S. Patent Publication 2002-0034764, the entirety of which is hereby incorporated by reference. The '372 application describes DBP-31 (now renamed GST-Omega-1) and DBP-32 (referred to in the literature as CLIC 1; Valenzuela et al., J. Biol. Chem., 272:12575-82 (1997); Tolk et al., Am. J. Physiol., 43:F1140-49 (1998)), both of which are DASU binding proteins and are related to GST-Omega-2 described herein.

### FIELD OF THE INVENTION

This invention relates to methods of using the newly identified diarylsulfonylurea (DASU) binding protein (DBP) GST-Omega-2, particularly as a novel therapeutic target for suppressing the release of inflammatory mediators such as interleukins IL-1 and IL-18.

### BACKGROUND OF THE INVENTION

Inflammatory diseases such as rheumatoid arthritis are characterized by an excessive production of cytokines that promote and/or maintain the inflammatory state. Prominent among them are IL-1 (both the □ and □ forms), tumor necrosis factor alpha (TNF□), and IL-18 (Dinarello, C. A. Blood 87:2095-2147 (1996); Aggarwal, B. B. and Natarajan, K. Eur Cytokine Netw. 7:93-124 (1996); Ushio, S. et al. J. Immunol. 156:4274-4279 (1996)). After release from producing cells, these cytokines bind to specific receptors on target cells to initiate cytokine signaling cascades. As a result of their importance in the disease process, therapeutic approaches aimed at regulating production and/or activity of these cytokines are desirable.

Most cytokines are secreted from cells via the constitutive secretory apparatus composed of the rough endoplasmic reticulum and Golgi apparatus, but IL-1 and IL-18 are exported by an atypical route (Rubartelli, A. et al. EMBO J. 9:1503-1510 (1990)). The need for this atypical export pathway is a consequence of the synthesis of IL-1 and IL-18 as polypeptides lacking signal sequences (Auron, P. E. et al. Proc. Natl. Acad. Sci. USA 81:7907-7911 (1984); March, C. J. et al. Nature 315:641-647 (1985); Ushio, S. et al. J. Immunol. 156:4274-4279 (1996)). This signal or leader sequence typically is found at the amino terminus of polypeptides that are destined to be released from the cell (von Heijne, G. J. Membrane Biol. 115:195-201 (1990)). A signal sequence serves as a molecular address to direct newly synthesized polypeptides into the endoplasmic reticulum. Because newly synthesized IL-1 (proIL-1) and IL-18 (proIL-18) lack this sequence, they accumulate within the cytoplasmic compartment of the producing cell. In addition to their co-localization, proIL-1□ and proIL-18 also must be processed by the protease caspase I (Thornberry, N. A. et al. Nature 356:768-774 (1992); Ghayur, T. et al. Nature 360:619-623 (1997)); this cleavage generates biologically active, mature forms of the cytokines competent to bind to target cell receptors. ProIL-1□ does not share this requirement for proteolytic activation (Moseley, B. et al. J. Biol. Chem. 262:2941-2944 (1987). Other polypeptides exported by similar non-traditional routes include: Mif-related protein (MRP) 8/14 (Rammes, A. et al. J. Biol. Chem. 272:9496-9502 (1997)), basic fibroblast growth factor (Abraham, J. A. Science 233: 545-548 (1986)), galectins (Cleves, A. E. et al. J. Cell Biol. 133:1017-1026 (1997)), a form of the IL-1 receptor antagonist (Haskill, S. et al. Proc. Natl. Acad. Sci. USA 88:3681-3685 (1991)), thioredoxin (Rubartelli, A. et al. J. Biol. Chem. 267:24161-24164 (1992)), and several virally-encoded polypeptides including VP22 and Tat (Ensoli, B. et al. J. Virol. 67:277-287 (1993)); Elliott, G. and O'Hare, P. Cell 88:223-233 (1997)).

Lipopolysaccharide (LPS)-treated monocytes and macrophages produce large quantities of proIL-1□, but the release of mature cytokine is inefficient in the absence of a secondary stimulus (Hogquist, K. A. et al. J. Immunol. 147:2181-2186 (1991)); Perregaux, D. et al. J. Immunol. 149:1294-1303 (1992)). Both proteolytic maturation of proIL-1□ and the release of mature cytokine are enhanced by treating LPS-activated cells with any of a number of different stimuli including: extracellular ATP, cytolytic T-cells, high concentrations of LPS, ionophore-like molecules, toxins, hypotonic stress, and mechanical stress (Hogquist, K. A. et al. Proc. Natl. Acad. Sci. USA 88:8485-8489 (1991); Perregaux, D. and Gabel, C. A. J. Biol. Chem. 269:15195-15203 (1994); Walev, I. et al. Eur Mol. Biol. Org. J. 14:1607-1614 (1995); Bhakdi, S. et al. J. Clin. Invest. 85:1746-1753 (1990); Chin, J. and Kostura, M. J. J. Immunol. 151:5574-5585 (1993)). Importantly, stimulus-coupled cytokine posttranslational processing is sensitive to pharmacological intervention. Thus, a variety of non-selective anion transport inhibitors such as ethacrynic acid and tenidap can block stimulus-coupled posttranslational processing of proIL-1□ (Laliberte, R. et al. J. Immunol. 153:2168-2179 (1994); Perregaux, et al. J. Immunol. 157:57-64 (1996); Perregaux et al. J. Immunol. 160:2469-2477 (1998)). These agents are effective inhibitors of IL-1 posttranslational processing independent of the nature of the activating stimulus. Moreover, their inhibitory effect is manifested as a complete suppression in the externalization of both IL-1□ and IL-1□.

A novel series of agents termed DASUs has been identified as potent inhibitors of stimulus-coupled posttranslational processing. These compounds are described and claimed in U.S. Pat. No. 6,166,064 (see also Perregaux et al., J. Pharmacol. Exp. Therap., 299(1):187-97 (2001)). Because IL-1 and IL-18 are important mediators of inflammation and inhibitors of their function provide therapeutic relief in animal models of disease (Cominelli, F. et al. J. Clin. Invest. 86:972-980 (1990); Akeson, A. L. et al. J. Biol. Chem. 271:30517-30523 (1996); Caron, J. P. et al. Arthritis Rheum. 39:1535-1544 (1996); Okamura, H. et al. Nature 378:88-91 (1995); Rothwell, N. J. Clin. Invest. 100:2648-2652 (1997)), agents that disrupt the process of stimulus-coupled posttranslational processing may be useful for the treatment in man and animals of disorders that are associated with inflammatory mediators. These include, for example, rheumatoid arthritis, osteoarthritis, asthma, inflammatory bowel disease, ulcerative colitis, neurodegeneration, atherosclerosis, and psoriasis.

This invention results from the identification of the protein and DNA sequences for a new DASU binding protein (DBP) that mediates the cytokine inhibitory activity of these agents. This new DBP may be used, for example, to screen for structurally unique drugs that disrupt stimulus-coupled posttranslational processing. Compounds that bind to this DBP also may be used as therapeutics in the treatment of inflammatory disorders.

This newly discovered DBP is a Glutathione S-transferase protein, named GST-Omega-2 (see Acc. No. XM 058395; Acc. No. CAC16040; and Ahmed et al., Biochim. Biophys. Acta, 1161:333-36 (1993)). It is given this name due to its homology with the previously described DBP named GST-Omega-1 (see '372 application cited hereinabove, as well as Board et al., J. Biol. Chem., 275(32):24798-806 (2000)).

### SUMMARY OF THE INVENTION

The present invention is directed to methods of screening for ligands that bind to GST-Omega-2 and that thereby inhibit the production of inflammatory cytokines, and thereby treat disease conditions having an inflammatory component. By practicing the methods of the present invention, new drugs will be found that will treat a variety of inflammatory diseases such as, for example, rheumatoid arthritis, osteoarthritis, asthma, inflammatory bowel disease, ulcerative colitis, neurodegeneration, atherosclerosis, and psoriasis.

In a first preferred embodiment, the present invention contemplates a method of screening for the ability of a test compound to inhibit the production of an inflammatory cytokine, said method comprising the steps of measuring the ability of said compound to bind to a polypeptide having the sequence of SEQ ID NO:2, a polypeptide having substantial homology to SEQ ID NO:2, a polypeptide coded for by the polynucleotide sequence of SEQ ID NO:1, or a polypeptide coded for by a polynucleotide sequence having substantial homology to SEQ ID NO:1; and determining that said test compound inhibits the production of an inflammatory cytokine if said test compound binds to said polypeptide.

In a second preferred embodiment, the present invention contemplates a method of treatment of a mammal having a disease condition having an inflammatory component comprising administering to said mammal a compound binding to a polypeptide having the sequence of SEQ ID NO:2, a polypeptide having substantial homology to SEQ ID NO:2, a polypeptide coded for by the polynucleotide sequence of SEQ ID NO:1, or a polypeptide coded for by a polynucleotide sequence having substantial homology to SEQ ID NO:1.

In a third preferred embodiment, the present invention contemplates a method of screening for the ability of a test compound to inhibit the production of an inflammatory cytokine, said method comprising the steps of measuring the ability of said compound to bind to a polypeptide having the sequence of SEQ ID NO:4, a polypeptide having substantial homology to SEQ ID NO:4, a polypeptide coded for by the polynucleotide sequence of SEQ ID NO:3, or a polypeptide coded for by a polynucleotide sequence having substantial homology to SEQ ID NO:3; and determining that said test compound inhibits the production of an inflammatory cytokine if said test compound binds to said polypeptide.

In a fourth preferred embodiment, the present invention contemplates a method of treatment of a mammal having a disease condition having an inflammatory component comprising administering to said mammal a compound binding to a polypeptide having the sequence of SEQ ID NO:4, a polypeptide having substantial homology to SEQ ID NO:4, a polypeptide coded for by the polynucleotide sequence of SEQ ID NO:3, or a polypeptide coded for by a polynucleotide sequence having substantial homology to SEQ ID NO:3.

In a fifth preferred embodiment, the present invention contemplates a method of screening for the ability of a test compound to treat a mammal having a disease condition having an inflammatory component, said method comprising the steps of measuring the ability of said compound to bind to a polypeptide having the sequence of SEQ ID NO:2, a polypeptide having substantial homology to SEQ ID NO:2, a polypeptide coded for by the polynucleotide sequence of SEQ ID NO:1, or a polypeptide coded for by a polynucleotide sequence having substantial homology to SEQ ID NO:1; and determining that said test compound treats a mammal having a disease condition having an inflammatory component if said test compound binds to said polypeptide.

In a sixth preferred embodiment, the present invention contemplates a method of screening for the ability of a test compound to treat a mammal having a disease condition having an inflammatory component, said method comprising the steps of measuring the ability of said compound to bind to a polypeptide having the sequence of SEQ ID NO:4, a polypeptide having substantial homology to SEQ ID NO:4, a polypeptide coded for by the polynucleotide sequence of SEQ ID NO:3, or a polypeptide coded for by a polynucleotide sequence having substantial homology to SEQ ID NO:3; and determining that said test compound treats a mammal having a disease condition having an inflammatory component if said test compound binds to said polypeptide.

### BRIEF DESCRIPTION OF THE DRAWINGS

FIG. 1 is a sequence alignment showing homologies at the amino acid level between the human and murine versions of GST-Omega-1 and GST-Omega-2.

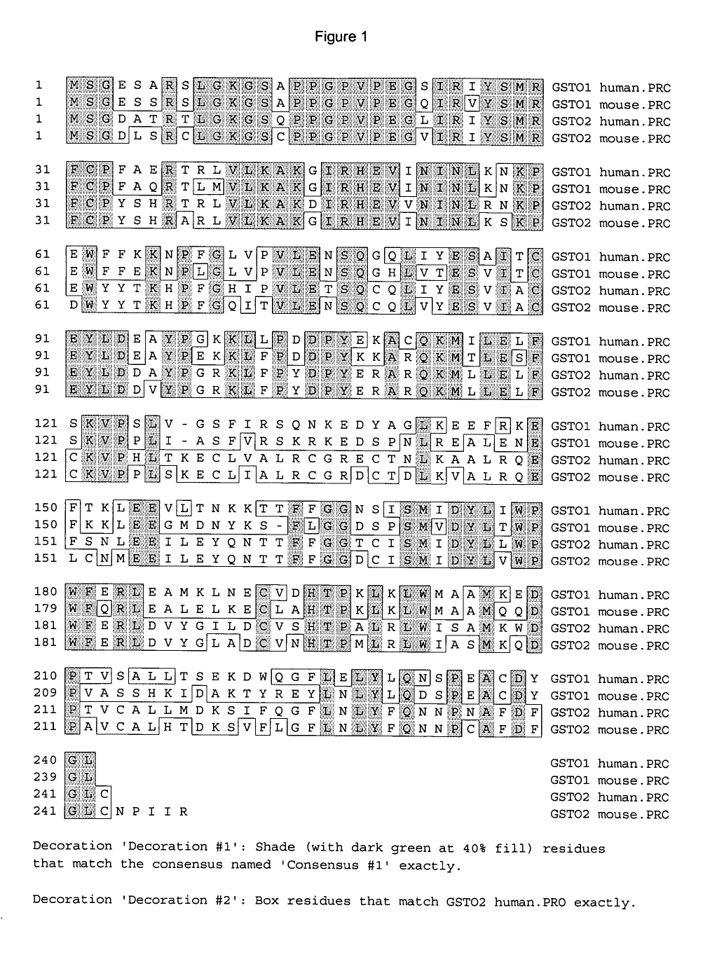

FIG. 2 is a sequence alignment showing homologies at the amino acid level between the human and murine versions of GST-Omega-2.

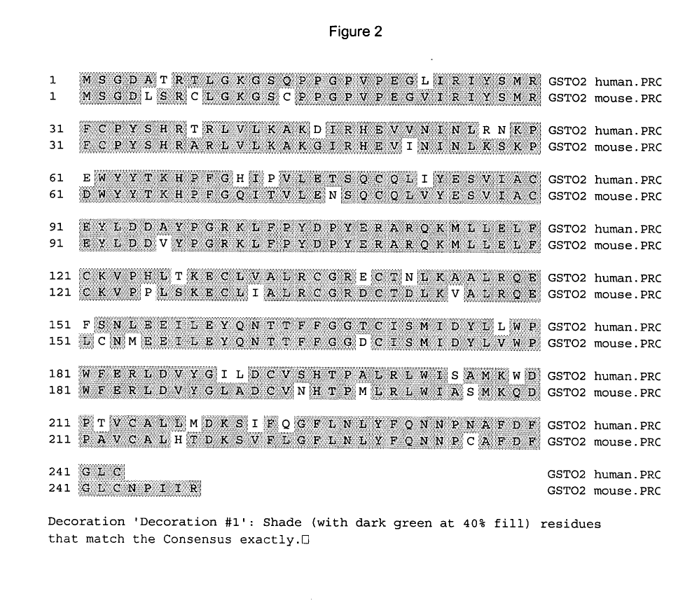

FIG. 3 is a sequence alignment showing homologies at the nucleotide level between the human and murine versions of GST-Omega-2.

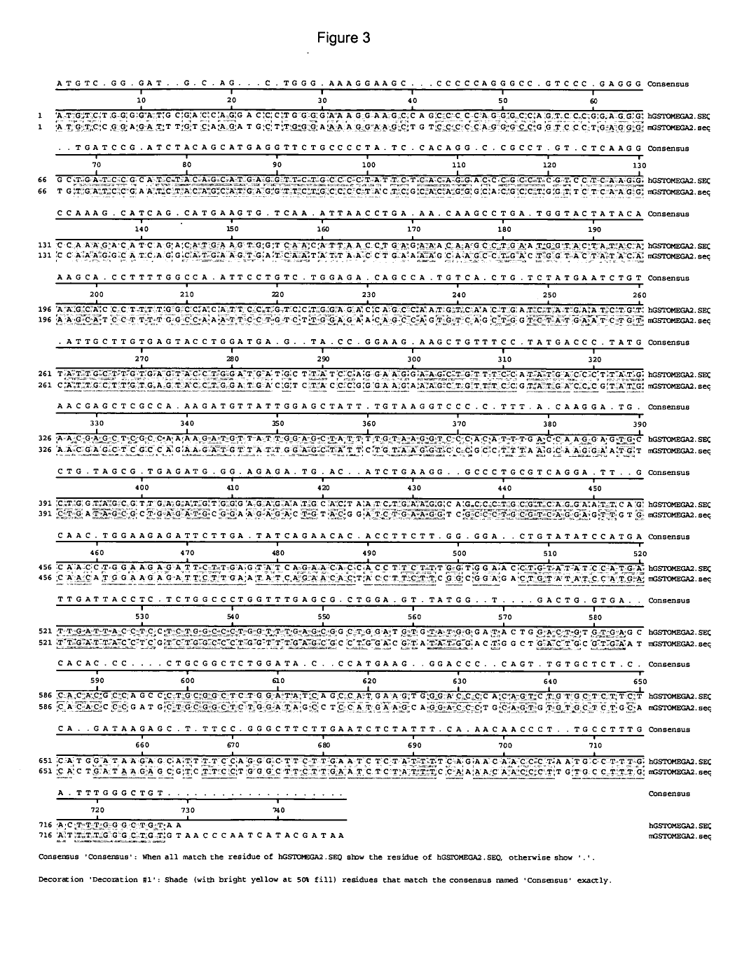

FIG. 4 illustrates the design of a vector employed to express GST-Omega-2 in insect cells.

### DETAILED DESCRIPTION OF THE INVENTION

The following abbreviations are used throughout this description and the appendant claims: ATP—adenosine triphosphate; Da—daltons; DASU—diarylsulfonylurea; DBP—diarylsulfonylurea binding protein; GST—glutathione S-transferase; HPLC—high performance liquid chromatography; IL—interleukin; IFNγ—interferon gamma; LPS—lipopolysaccharide; PAGE—polyacrylamide gel electrophoresis; r—recombinant; SDS—sodium dodecyl sulfate; and TNF\tilde{□}tumor necrosis factor alpha.

By “administer” or “administration”, when referring to administering a compound is meant that the compound is delivered to a patient in a manner that allows the compound to reach the necessary parts of the patient's anatomy so as to be effective in treating a disease condition having an inflammatory component. In some cases systemic administration, such as by oral dosage or intravenously, will be acceptable. In other cases it may be necessary to administer the compound at the site of the inflammatory reaction, such as by localized application of a cream, or localized injection of the compound at the reactive site on the patient's body.

By “substantial homology” is meant that the polynucleotide sequences being compared have at least about 80% homology. By “high homology” is meant that the polynucleotide sequences being compared have at least about 90% homology. By “very high homology” is meant that the polynucleotide sequences being compared have at least about 95% homology. When referring to polypeptide/protein/amino acid sequences, substantial homology, high homology, and very high homology also mean at least about 80%, 90%, and 95% homology, respectively. The homologies referred to throughout this application are calculated using the BLAST algorithm (Basic Local Alignment Search Tool (Altschul, J. Mol. Evol., 36:290-300 (1993); Altschul et al., J. Mol. Biol., 215:403-410 (1990)) for both nucleotide and amino acid sequence homologies. “Homology” and “identity” may be used interchangeably when referring to the level of similarity of nucleotide or amino acid sequences.

Another way to indicate relative homologies of polynucleotide sequences is by hybridization. It is possible to determine relationships among polynucleotide sequences based upon their abilities to hybridize to one another under specific conditions of solvent, salt concentrations, and temperatures (among other variables). In most cases, hybridization reactions are performed under high stringency conditions, for example, conditions where the salt concentration is no more than about 1 molar (M) and the temperature is at least about 25° C., e.g., 750 millimolar (mM) sodium chloride (NaCl), 50 mM sodium phosphate (NaPhosphate), and 5 mM ethylenediaminetetraacetic acid (EDTA), at pH 7.4 (5×SSPE) and a temperature of from about 25° C. to about 30° C.

By “bind” or “binding” is meant the ability of one molecule to specifically but non-covalently attach itself to another molecule. Binding does not include non-specific non-covalent reactions between molecules, nor does it include covalent reactions. Binding is analogous to hybridization, though hybridization typically only refers to polynucleotide binding, while binding is generally used for binding between any other two types of molecules. Binding includes the non-covalent component of the reaction that occurs between a compound such as Compound 2 (see Example 4, infra), which compound is capable of non-covalently associating with another molecule, even though such close association results in the creation of a covalent bond between the epoxide group of Compound 2 and the molecule with which it associates. In other words, the fact that the compounds eventually become covalently bound does not detract from the non-covalent character of the binding that can also occur therebetween.

By “inhibit” is meant a statistically significant decline in a measured event, as measured by the student T-test, or any other suitable statiscal method.

By “inflammatory cytokine” is meant any of a number of hormone-like low molecular weight polypeptides, secreted by many different cell types, and which are involved in the regulation of the intensity and duration of immune responses, as well as being involved in cell-to-cell communication. In particular, inflammatory cytokines refer to those cytokines that can cause or perpetuate an inflammatory response in any tissue. Exemplary inflammatory cytokines include IL-1, IL-18, IL-6, TNFα, IFNγ.

As described in the '372 application, DASUs inhibit stimulus-coupled IL-1□ posttranslational processing, and therefore must bind to one or more cellular proteins involved in the cytokine export pathway. To identify these proteins, radiolabeled DASU analogs were synthesized containing an epoxide group that could react with functional groups on proteins to form covalent, irreversible adducts. Treatment of human monocytes continuously with these agents resulted in a dose-dependent inhibition in ATP-induced IL-1□ production.

DBPs allow screening for specific drugs that may represent improved therapeutics for use in treating inflammatory disorders. Further work has now led to the discovery of a new DBP, named GST-Omega-2. GST-Omega-2, and/or the DNA encoding it, can be used in vitro to establish assays for measuring the potency of compounds binding to this polypeptide.

Agents that bind to GST-Omega-2 have utility as inhibitors of the production of cytokines such as IL-1 and IL-18 that are externalized by non-traditional stimulus-coupled posttranslational processing. Administration of these agents to mammals thus constitutes a method to treat disorders characterized by the overproduction of inflammatory cytokines. These disorders include rheumatoid arthritis, osteoarthritis, inflammatory bowel disease, ulcerative colitis, neurodegeneration, stroke, sepsis, atherosclerosis, asthma, and the like.

The compounds that bind to DBP's can also be used to produce pharmaceutical compositions.

Given the teachings of the present application, those of skill in the art can readily produce sufficient amounts of GST-Omega-2 for use in the methods of the present invention. Any of a variety of well-known methods of producing recombinant proteins may be used to produce GST-Omega-2 in the quantity desired (see, e.g., Example 5). Additionally, those of skill in the art will appreciate that any of a variety of methods may be used to purify the GST-Omega-2 for use in the methods of the present invention (see, e.g., Example 4).

In one embodiment, the present invention encompasses methods of screening for the ability of a compound to inhibit the production of an inflammatory cytokine comprising determining the ability of said compound to bind to a polypeptide coded for by the polynucleotide sequence of SEQ ID NO:1, the nucleotide sequence of human GST-Omega-2. In a related embodiment, the invention method encompasses the use of a polypeptide having the sequence of SEQ ID NO:2, the amino acid sequence of human GST-Omega-2. In further related embodiments, the invention method encompasses the use of a polypeptide coded for by the polynucleotide sequence of SEQ ID NO:3, or a polypeptide having the sequence of SEQ ID NO:4, which are respectively the nucleotide and amino acid sequences of murine GST-Omega-2. All of these methods are useful for the discovery of compounds that inhibit the production of cytokines, and to treat disorders characterized by the overproduction of inflammatory cytokines.

The invention also encompasses methods of using GST-Omega-2 variants. A preferred GST-Omega-2 variant is one having at least 80%, more preferably at least 90%, and even more preferably at least 95% amino acid sequence homology to the GST-Omega-2 amino acid sequence (SEQ ID NO:2 or SEQ ID NO:4) and which retains at least one biological, immunological or other functional characteristic or activity of GST-Omega-2.

The invention also encompasses methods of using polynucleotides that encode GST-Omega-2. Accordingly, any nucleic acid sequence which encodes the amino acid sequence of GST-Omega-2 can be used to produce recombinant molecules which express GST-Omega-2. In a particular embodiment, the invention encompasses the use of the polynucleotide comprising the nucleic acid sequence of SEQ ID NO:1 or SEQ ID NO:3. The invention also encompasses methods of using GST-Omega-2 variants. A preferred GST-Omega-2 variant is one having at least 80%, more preferably at least 90%, and even more preferably at least 95% nucleotide sequence identity to the GST-Omega-2 amino acid sequence (SEQ ID NO:1 or SEQ ID NO:3) and which encode a protein retaining at least one biological, immunological, or other functional characteristic or activity of GST-Omega-2. Also encompassed by the invention are methods of using polynucleotide sequences that are capable of hybridizing to the nucleotide sequences shown in SEQ ID NO:1 and SEQ ID NO:3, under various conditions of stringency, particularly high stringency conditions, and which encode a protein retaining at least one biological, immunological, or other functional characteristic or activity of GST-Omega-2.

It will be appreciated by those skilled in the art that as a result of the degeneracy of the genetic code, a multitude of nucleotide sequences encoding GST-Omega-2, some bearing minimal homology to the nucleotide sequences of any known and naturally occurring gene, may be produced. Thus, the invention contemplates each and every possible variation of nucleotide sequence that could be made by selecting combinations based on possible codon choices. These combinations are made in accordance with the standard triplet genetic code as applied to the nucleotide sequence of naturally occurring GST-Omega-2, and all such variations are to be considered as being specifically disclosed.

Although nucleotide sequences which encode GST-Omega-2 and its variants are preferably capable of hybridizing to the nucleotide sequence of the naturally occurring GST-Omega-2 under appropriately selected conditions of stringency, it may be advantageous to produce nucleotide sequences encoding GST-Omega-2 or its derivatives possessing a substantially different codon usage. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding GST-Omega-2 and its derivatives without altering the encoded amino acid sequences include the production of RNA transcripts having more desirable properties, such as a greater half-life, than transcripts produced from the naturally occurring sequence.

In a preferred embodiment, the GST-Omega-2 polypeptide is used to screen compounds and discover those compounds that bind thereto. Such compounds are likely to impact the activity of the GST-Omega-2 protein and thereby inhibit the production of cytokines in vivo. Methods for measuring the binding of a test compound to a protein are numerous and well known to those of skill in the art, and include both competitive and non-competitive assays. Good reference books for such binding assays include Principles of Competitive Protein-Binding Assays, William D. Odell (Editor), John Wiley & Sons, Second edition (January 1983) and Quantitative Characterization of Ligand Binding, Donald J. Winzor and William H. Sawyer, John Wiley & Sons (October 1995).

Once it is determined that a test compound is a ligand for GST-Omega-2, the test compound can be further analyzed for its suitability as a human or animal pharmaceutical drug. The compounds may be administered to animals (including humans) orally, parenterally, topically, or otherwise, in any conventional form of preparation, such as capsules, microcapsules, tablets, granules, powder, troches, pills, suppositories, injections, suspensions, syrups, and the like.

The compounds can be prepared for pharmaceutical administration by methods commonly employed using conventional additives, such as excipients (e.g., sucrose, starch, mannitol, sorbitol, lactose, glucose, cellulose, talc, calcium phosphate, calcium carbonate, and the like), binders (e.g., cellulose, methylcellulose, hydroxymethylcellulose, polypropylpyrrolidone, polyvinylprrolidone, gelatin, gum arabic, polyethyleneglycol, sucrose, starch, and the like), disintegrators (e.g., starch, carboxymethylcellulose, hydroxypropylstarch, low substituted hydroxypropylcellulose, sodium bicarbonate, calcium phosphate, calcium citrate, and the like), lubricants (e.g., magnesium stearate, light anhydrous silicic acid, talc, sodium lauryl sulfate, and the like), flavoring agents (e.g., citric acid, menthol, glycine, orange powder, and the like), preservatives (e.g., sodium benzoate, sodium bisulfite, methylparaben, propylparaben, and the like), stabilizers (e.g., citric acid, sodium citrate, acetic acid, and the like), suspending agents (e.g., methylcellulose, polyvinylpyrrolidone, aluminum stearate, and the like), dispersing agents (e.g., hydroxypropylmethylcellulose and the like), diluents (e.g., water, alcohol, glycerin, and the like), and base waxes (e.g., cocoa butter, white petrolatum, polyethylene glycol, and the like).

The compounds may be administered once a day or in multiple daily doses, with a preferred daily dosage of about 0.001 to about 100 mg in adult human patients. This dosage may be properly varied depending on the age, body weight, and medical condition of the patient, as well as the mode of administration, as may be determined by those of skill in the art. A more preferred daily dose is about 1.0 to about 10 mg in human patients. One dose per day is preferred. Controlled release, sustained release, and/or delayed release oral or parenteral compositions may be used.

The present invention will now be further described with the following examples that are representative of aspects of the invention, but which should not be read as limiting to the scope of the appendant claims.

### EXAMPLES

### Example 1

### IL-1□ Production Assay—ELISA Method

Human monocytes were isolated from heparinized blood collected from normal volunteers. 100 ml of blood was diluted with 20 ml of RPMI 1640 medium that contained 5% FBS, 25 mM Hepes, pH 7.3, 2 mM glutamine, and 1% penicillin/streptomycin (Maintenance Medium). 30 ml of this suspension then was layered onto 15 ml of Lymphocyte Separation Medium (Organo Technicon) and centrifuged for 30 min at 1400 rpm at room temperature in a clinical centrifuge. The resulting mononuclear cell layer was harvested, diluted with Maintenance Medium, and the cells were pelleted by centrifugation. The recovered cells were washed with Maintenance Medium by repeated centrifugation. Each well of a 96 well plate was seeded with 2×10⁵ mononuclear cells in 0.1 ml of Maintenance Medium; monocytes were allowed to adhere for 2 hr, then the medium (containing non-adherent cells) was replaced with 0.1 ml of fresh medium, and the monocytes were incubated overnight at 37° C. Monocytes were activated with 10 ng/ml of LPS (E. coli; serotype 055:B5) for 2 hr after which the medium was replaced with 0.1 ml of Release Medium (RPMI 1640 that contained 20 mM Hepes, pH 6.9, 100 units/ml penicillin, 100 μg/ml streptomycin, and 1% FBS) containing the indicated concentration of test agent. Test agents were dissolved in DMSO and the final DMSO concentration in all wells was maintained at 0.2%. After a 15-min incubation, 2 mM ATP was introduced into each well (11 μl of 20 mM ATP previously adjusted to pH 7) and the cultures were incubated for an additional 3 hr at 37° C. Cells subsequently were removed by centrifugation, media supernatants were harvested, and IL-1□ levels within the media samples were determined with a specific ELISA kit (R and D Systems). See also Perregaux et al., J. Immunol., 168:3024-32 (2002).

### Example 2

### IL-1□ Production Assay—Metabolic Method

Human mononuclear cells were seeded (1×10⁷ cells/well) into 6-well cluster dishes in 2 ml of RPMI 1640 medium containing 5% FBS and 25 mM Hepes, pH 7.3. Monocytes were allowed to adhere for 2 hr after which the supernatants were discarded and the attached cells were rinsed once with 2 ml of Maintenance medium. The attached monocytes were incubated overnight at 37° C. in a 5% CO₂ environment. Cells were activated with LPS (10 ng/ml) for 2 hr, then labeled for 60 min in 1 ml of methionine-free RPMI 1640 medium containing 1% dialyzed FBS, 25 mM Hepes, pH 7.2, and 83 μCi/ml of [³⁵S]methionine (Amersham Corp., Malvern Pa., 1000 Ci/mmol). The labeled cells were rinsed with 2 ml Release Medium, after which 1 ml of Release Medium containing test agent was added and the cells were incubated for 15 to 60 min at 37° C. At this point fresh Release Medium containing 2 mM ATP (in the absence or presence of the test agent) was added to initiate cytokine posttranslational processing.

Cells and media were separated after 3 hr of stimulus-induced processing and the media were clarified by centrifugation to remove cells and/or cell debris; the resulting supernatants were harvested and adjusted to 1% in Triton X-100, 0.1 mM PMSF, 1 mM iodoacetic acid, 1 μg/ml pepstatin, and 1 μg/ml leupeptin by addition of concentrated stock solutions of these reagents. Adherent monocytes were solubilized by addition of 1 ml of an extraction buffer composed of 25 mM Hepes, pH 7, 1% Triton X-100, 150 mM NaCl, 0.1 mM PMSF, 1 mM iodoacetic acid, 1 μg/ml pepstatin, 1 μg/ml leupeptin, and 1 mg/ml ovalbumin; 50 μl of this extraction buffer also was added to the pellets obtained after clarification of the media supernatants and these samples were combined with the corresponding cell extract. After a 30 min incubation on ice, both the media and cell extracts were clarified by centrifugation at 45,000 rpm for 30 min in a Beckman table top ultracentrifuge using a TLA 45 rotor (Beckman Instruments, Palo Alto, Calif.).

IL-1□ was immunoprecipitated from detergent extracts of cell and media samples by addition of 3 μl of a rabbit anti-human IL-1□ serum (Collaborative Biochemical Products Bedford, Mass.). After a 2-hr incubation at 4° C., 0.25 ml of a 10% suspension of Protein A-Sepharose (Sigma) was added to each tube and the resulting immune complexes were recovered by centrifugation. The bead-bound complexes were washed 5 times with 10 mM Tris, pH 8, 10 mM EDTA, 1% Triton X-100, 0.4% deoxycholate, 0.1% SDS and once with 50 mM Tris, pH 6.8. The final pellets were suspended in 0.1 ml of disaggregation buffer and boiled for 3 min; beads were removed by centrifugation and the disaggregated immunoprecipitate supernatants were stored at −20° C. prior to analysis by SDS gel electrophoresis and autoradiography. Gels were soaked in Amplify (Amersham) prior to drying. Quantitation of the amount of radioactivity associated with the various species of IL-1□ was determined with the use of an Ambis Image Analysis System (San Diego, Calif.) or by phosphorimager analysis.

Murine macrophages isolated by peritoneal lavage of Balb/c mice were seeded into 6 well dishes (precoated with Natrix); 1×10⁶ cells were added per well in 2 ml of RPMI 1640 medium containing 5% FBS. After an overnight incubation, these cells were stimulated with LPS (1 μg/ml for 75 min) and then labeled for 60 min with [³⁵S]methionine (80 μCi) in 1 ml of methionine-free minimal essential medium containing 25 mM Hepes, 1 μg/ml LPS, and 1% dialyzed FBS. Labeled cells were washed with RPMI 1640, 25 mM Hepes, pH 7.3, containing 1 μg/ml LPS and 1% FBS after which 1 ml of the same medium containing allogeneic cytotoxic T lymphocytes was introduced in the absence or presence of Compound 3 (1-(4-chloro-2,6-diisopropyl-phenyl)-3-[3-(1-hydroxy-ethyl)-benzenesulfonyl]-urea; an agonist of DBPs as described in U.S. Patent Publication 2002-0034764); a lymphocyte to macrophage ratio of 20 to 1 was employed. The cytolytic T lymphocyte preparation was prepared in advance by establishing an in vitro mixed lymphocyte reaction between C57/BI mice effector spleen cells stimulated with irradiated target spleen cells obtained from Balb/c mice as detailed previously (Perregaux et al. J. Immunol. 157:57-64 (1996)). After 4 hr of co-culture the media were harvested and cleared of cells and/or cell debris by centrifugation, and released IL-1□ was recovered by immunoprecipitation as detailed above. Immunoprecipitates were analyzed by SDS gel electrophoresis and the quantity of radioactivity associated with the 17 kDa IL-1□ species was determined by Ambis Image analysis. See also Perregaux et al., J. Immunol., 168:3024-32 (2002).

### Example 3

### In vivo IL-1□ Production Assay

Mice were given an i.p. injection of 1 μg of LPS in 0.5 ml of PBS. One hour later Compound 3 was dosed orally in 0.5% methylcellulose. After a further one hour, an i.p. injection of ATP disodium salt (30 mM) in 0.5 ml of PBS, was administered; the pH of the ATP solution was adjusted to 7.3 prior to injection. Fifteen minutes later, the mice were euthanized by cervical dislocation and the peritoneal cavity lavaged with 3 ml of ice cold PBS containing 10 units/ml of heparin sodium salt, 0.25 mM phenylmethysulfonyl fluoride, 1 μg/ml leupeptin, 1 μg/ml pepstatin, and 1 mM EDTA. IL-1□ and IL-1□ in the lavage fluid were measured using commercially available ELISA kits from Endogen (IL-1□) and Genzyme (IL-1□). See also Perregaux et al., J. Pharmacol. Exp. Therap., 299(1):187-97 (2001).

### Example 4

### GST-Omega-2 Binds to a Diarylsulfonylurea Affinity Column

Human embryonic kidney (HEK293) cells were transfected with a pcDNA3.1 mammalian expression vector encoding FLAG-tagged human GST-Omega-2. Stable transfectants isolated by growth in the presence of G418 were subcloned and a high expressing colony was identified. Four confluent 10 cm dishes of this cell line were established after which the cells were harvested and then disrupted by nitrogen cavitation. The resulting cell lysate was clarified by centrifugation and the supernatant fraction was recovered and applied to an affinity column (1 ml) composed of 1-(1,2,3,5,6,7-Hexahydro-s-indacen-4-yl)-3-[2-fluoro-5-oxiranylbenzenesulfonyl]-urea (hereinafter referred to as Compound 2 as per U.S. Patent Publication 2002-0034764) attached to Thiolpropyl-Sepharose. Compound 2 is a diarylsulfonylurea that contains an epoxide group which can react to form a covalent linkage with Thiolpropyl-Sepharose. In some cases, the cell extract was applied to the column in a buffer containing 5 mM of a competitive inhibitor, 1-(1,2,3,5,6,7-Hexahydro-s-indacen-4-yl)-3-[4-(1-hydroxy-1-methylethyl)-furan-2-sulfonyl]-urea (hereinafter referred to as Compound 4 as per the '372 application). The column was washed with buffer then eluted with a denaturing detergent, sodium dodecyl sulfate (SDS). Individual 1 ml fractions were collected and aliquots of each were fractionated by SDS/PAGE. The separated polypeptides were transferred to nitrocellulose for Western analysis; the combination of an anti-Flag M2 antibody and goat anti-mouse HRP secondary antibody was employed for detection of the recombinant polypeptide.

When the elution buffer did not contain Compound 4, the Western blot of the column fractions did not show passage of the flag-tagged GST-Omega-2. However, elution with SDS did result in column fractions containing the flag-tagged GST-Omega-2. Thus, it is clear that GST-Omega-2 binds to the diarylsulfonylurea affinity column and is elutable with SDS. Contrarily, when the elution buffer contained 5 mM Compound 4, the Western blot of column fractions did contain the flag-tagged GST-Omega-2, indicating that Compound 4 competed with the specific binding of GST-Omega-2 to the diarylsulfonylurea affinity column.

### Example 5

### Design of Vector Employed to Express GST-Omega-2 in Insect Cells

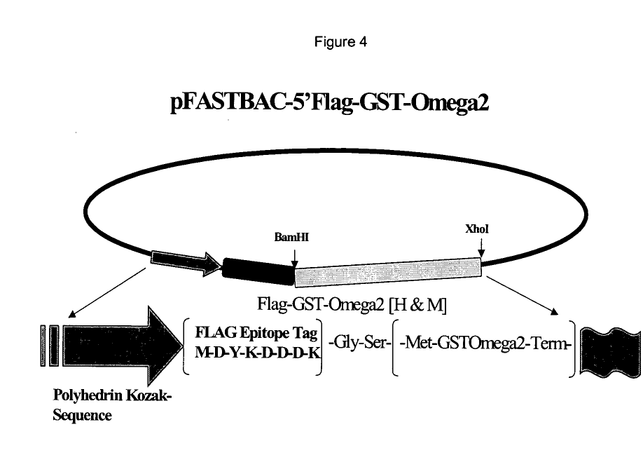

The pFASTBAC-5 vector was obtained from Gibco and re-engineered to encode an N-terminal FLAG-tag upstream of the multiple cloning site. The coding sequence for GST-Omega-2 was re-amplified using polymerase chain reaction building on the required BamH1 and Xho1 restriction sites necessary for in-frame insertion of this cDNA into the pFASTBAC-flag shuttle vector. This vector was employed in the generation of viral populations for the subsequent expression of GST-Omega-2 in SF9 insect cells. See FIG. 4 for a diagram of this vector.

### Example 6

### GST-Omega-2 is Selectively Labeled by [¹⁴C]Compound 2

Insect cells (SF9) were infected with a baculovirus construct encoding flag-tagged human GST-Omega-2 (see Example 5) and seeded into individual 10 cm dishes. For comparison, SF9 cells also were infected with a baculovirus construct encoding IKK□ (I-κB kinase-γ/NF-κB essential modulator (NEMO)). After 48 hr of infection, media were removed and replaced with fresh medium (6 ml) containing from 0.01 to 10 μM of [¹⁴C]Compound 2. Cells were incubated with the radiolabeled diarylsulfonylurea for 60 min (at room temperature) and then collected by centrifugation and washed to remove unbound test agent. Each cell pellet then was suspended in 5 ml of PBS and divided into 2 fractions. Cells within one of these fractions (⅕ of the total) were collected by centrifugation and suspended in 150 □l of SDS/PAGE disaggregation buffer. Cells within the other fraction (⅘ of the total) were collected by centrifugation, solubilized by detergent extraction, and recombinant GST-Omega-2 was recovered by immunoprecipitation with the anti-Flag M2 antibody. Both the total cell lysates and the immunoprecipitates were fractionated by SDS/PAGE and the dried gels were analyzed by autoradiography.

At the highest test concentration (10 □M), [¹⁴C]Compound 2 appeared to incorporate into several polypeptides present within the control (IKK□-expressing) cells, and these same polypeptides were detected within the GST-Omega-2-expressing cells. However, the latter also contained a polypeptide that co-migrated with a standard of GST-Omega-2 and which was not observed in the IKK□-expressing cells. When 1 □M [¹⁴C]Compound 2 was applied to the infected cells, only the polypeptide migrating as GST-Omega-2 appeared labeled. Immunoprecipitation with the anti-flag antibody confirmed that the [¹⁴C]Compound 2-labeled protein was indeed due to the flag-tagged GST-Omega-2; a dose-dependent increase in the incorporation of radioactivity into the immunoprecipitated protein was observed.

### Example 7

### Competitive Binding Assay

Insect cells (SF9) were infected with a baculovirus construct encoding flag-tagged human GST-Omega-2 (see Example 5) and seeded into individual 10 cm dishes. After 48 hr of infection, media were removed and replaced with fresh medium (6 ml) containing 100, 200 or 400 μM Compound 4, 10 μM Compound 2, or neither compound. The cultures were incubated for 30 min at room temperature after which 2 □M [¹⁴C]Compound 2 was introduced and the cultures were incubated for 60 min at room temperature. The cells then were harvested by centrifugation, washed with PBS to remove unbound radioactivity, and solubilized by detergent extraction. GST-Omega-2 was recovered by immunoprecipitation with anti-Flag M2 antibody, and the resulting immunoprecipitates were fractionated by SDS/PAGE; the dried gel subsequently was analyzed by phosphorimager analysis.

Relative to the control cultures, the presence of Compound 4 dose-dependently inhibited incorporation of [¹⁴C]Compound 2 into GST-Omega-2. Likewise, unlabeled Compound 2 blocked incorporation of [¹⁴C]Compound 2 into GST-Omega-2. Compound 4 inhibited incorporation by 63%, 73% and 84% at 100 □M, 200 □M and 400 □M, respectively. Unlabeled Compound 2 inhibited incorporation by 90% at 10 □M.

### Example 8

### Mass Analysis of Recombinant Flag-Tagged Human GST-Omega-2

Mass spectrometry of the affinity-purified recombinant protein indicated a mass of 29564±3 Da, a value approximately 40 Da higher than the theoretical mass for a polypeptide with the expected amino acid sequence. This discrepancy was resolved when peptide mapping of a tryptic digest of the recombinant protein by HPLC and mass spectrometry showed that the amino-terminal peptide was □-N-acetylated. Acetylation, a relatively common modification of proteins expressed in higher eukaryotic cells, adds 42 Da of mass. The results indicated that recombinant GST-Omega-2 had the expected amino acid sequence, with the post-translational addition of an □-N-acetyl group.

### Example 9

### Murine GST-Omega-2 Also is Selectively Labeled by [¹⁴C]Compound 2

Insect cells (SF9) were infected with a baculovirus construct encoding flag-tagged murine GST-Omega-2 (see Example 5) and seeded into individual 10 cm dishes. For comparison, SF9 cells also were infected with a baculovirus construct encoding IKK□ (IkappaB kinase gamma). After 48 hr of infection, media were removed and replaced with fresh medium (6 ml) containing from 0.01 to 10 μM [¹⁴C]Compound 2. Cells were incubated with the radiolabeled diarylsulfonylurea for 60 min (at room temperature) and then collected by centrifugation and washed to remove unbound test agent. Each cell pellet then was suspended in 5 ml of PBS and divided into 2 fractions. Cells within one of these fractions (⅕ of the total) were collected by centrifugation and suspended in 150 □l of SDS/PAGE disaggregation buffer. Cells within the other fraction (⅘ of the total) were collected by centrifugation, solubilized by detergent extraction, and recombinant GST-Omega-2 was recovered by immunoprecipitation with the anti-Flag M2 antibody. Both the total cell lysates and the immunoprecipitates were fractionated by SDS/PAGE and the dried gels were analyzed by autoradiography.

At the highest test concentration (10 □M), [¹⁴C]Compound 2 appeared to incorporate into several polypeptides present within the control (IKK□-expressing) cells, and these same polypeptides were detected within the GST-Omega-2-expressing cells. However, the latter also contained a polypeptide that co-migrated with a standard of GST-Omega-2 and which was not observed in the IKK□-expressing cells. When 1 □M [¹⁴C]Compound 2 was applied to the infected cells, only the polypeptide migrating as GST-Omega-2 appeared labeled. Immunoprecipitation with the anti-flag antibody confirmed that the [¹⁴C]Compound 2-labeled protein was indeed due to the flag-tagged GST-Omega-2; a dose-dependent increase in the incorporation of radioactivity into the immunoprecipitated protein was observed.

All patents and publications (of any sort, including sequences cited by accession number) mentioned in the above specification are herein incorporated by reference in their entirety. Various modifications and variations of the described invention will be apparent to those skilled in the art without departing from the scope and spirit of the invention. Although the invention has been described in connection with specific preferred embodiments, it should be understood that the invention as claimed is not limited to such specific embodiments.

1. A method of screening for the ability of a test compound to inhibit the production of an inflammatory cytokine, said method comprising the steps of:
a) measuring the ability of said compound to bind to a polypeptide having the sequence of SEQ ID NO:2, a polypeptide having substantial homology to SEQ ID NO:2, a polypeptide coded for by the polynucleotide sequence of SEQ ID NO:1, or a polypeptide coded for by a polynucleotide sequence having substantial homology to SEQ ID NO:1; and
b) determining that said test compound inhibits the production of an inflammatory cytokine if said test compound binds to said polypeptide.

2. The method of claim 1 wherein said inflammatory cytokine is IL-1 or IL-18.

3. The method of claim 1 wherein said polypeptide has high homology to SEQ ID NO:2, and said polynucleotide sequence has high homology to SEQ ID NO:1.

4. A method of treatment of a mammal having a disease condition having an inflammatory component comprising administering to said mammal a compound binding to a polypeptide having the sequence of SEQ ID NO:2, a polypeptide having substantial homology to SEQ ID NO:2, a polypeptide coded for by the polynucleotide sequence of SEQ ID NO:1, or a polypeptide coded for by a polynucleotide sequence having substantial homology to SEQ ID NO:1.

5. The method of claim 4 wherein said polypeptide has high homology to SEQ ID NO:2, and said polynucleotide sequence has high homology to SEQ ID NO:1.

6. A method of screening for the ability of a test compound to inhibit the production of an inflammatory cytokine, said method comprising the steps of:
a) measuring the ability of said compound to bind to a polypeptide having the sequence of SEQ ID NO:4, a polypeptide having substantial homology to SEQ ID NO:4, a polypeptide coded for by the polynucleotide sequence of SEQ ID NO:3, or a polypeptide coded for by a polynucleotide sequence having substantial homology to SEQ ID NO:3; and
b) determining that said test compound inhibits the production of an inflammatory cytokine if said test compound binds to said polypeptide.

7. The method of claim 6 wherein said inflammatory cytokine is IL-1 or IL-18.

8. The method of claim 6 wherein said polypeptide has high homology to SEQ ID NO:4, and said polynucleotide sequence has high homology to SEQ ID NO:3.

9. A method of treatment of a mammal having a disease condition having an inflammatory component comprising administering to said mammal a compound binding to a polypeptide having the sequence of SEQ ID NO:4, a polypeptide having substantial homology to SEQ ID NO:4, a polypeptide coded for by the polynucleotide sequence of SEQ ID NO:3, or a polypeptide coded for by a polynucleotide sequence having substantial homology to SEQ ID NO:3.

10. The method of claim 9 wherein said polypeptide has high homology to SEQ ID NO:4, and said polynucleotide sequence has high homology to SEQ ID NO:3.

11. A method of screening for the ability of a test compound to treat a mammal having a disease condition having an inflammatory component, said method comprising the steps of:
a) measuring the ability of said compound to bind to a polypeptide having the sequence of SEQ ID NO:2, a polypeptide having substantial homology to SEQ ID NO:2, a polypeptide coded for by the polynucleotide sequence of SEQ ID NO:1, or a polypeptide coded for by a polynucleotide sequence having substantial homology to SEQ ID NO:1; and
b) determining that said test compound treats a mammal having a disease condition having an inflammatory component if said test compound binds to said polypeptide.

12. A method of screening for the ability of a test compound to treat a mammal having a disease condition having an inflammatory component, said method comprising the steps of:
a) measuring the ability of said compound to bind to a polypeptide having the sequence of SEQ ID NO:4, a polypeptide having substantial homology to SEQ ID NO:4, a polypeptide coded for by the polynucleotide sequence of SEQ ID NO:3, or a polypeptide coded for by a polynucleotide sequence having substantial homology to SEQ ID NO:3; and
b) determining that said test compound treats a mammal having a disease condition having an inflammatory component if said test compound binds to said polypeptide.
